(19) United States
(12) Patent Application Publication
(10) Pub. No.: US 2019/0391147 A1
(43) Pub. Date: Dec. 26, 2019

(54) METHODS AND KITS FOR DETECTING MYO/NOG CELLS IN HUMAN ADIPOSE TISSUE AND METHODS OF USE THEREOF

(71) Applicant: ROWAN UNIVERSITY, GLASSBORO, NJ (US)

(72) Inventors: MINDY GEORGE-WEINSTEIN, GLASSBORO, NJ (US); SPENCER BROWN, GLASSBORO, NJ (US); JACQUELYN GERHART, GLASSBORO, NJ (US)

(21) Appl. No.: 16/090,056

(22) PCT Filed: Apr. 7, 2017
(86) PCT No.: PCT/US17/26606

§ 371 Date: Sep. 28, 2018

Related U.S. Application Data
(60) Provisional application No. 62/319,553, filed on Apr. 7, 2016.

(51) Int. Cl. G01N 33/569; C12N 5/074; A61K 35/28; A61K 45/06; A61K 9/00

(52) U.S. Cl. CPC G01N 33/56966; C12N 5/0607; A61K 9/0014; A61K 45/06; A61K 35/28

(57) ABSTRACT

The present invention relates to methods and kits for detecting Myo/Nog cells in a sample containing human adipose tissue, as well as therapeutic use thereof for tissue regeneration.

### I. CROSS-REFERENCE TO RELATED APPLICATIONS

This application is a 35 U.S.C. § 371 national phase application of, and claims priority to, International Application No. PCT/US2017/026606, filed Apr. 7, 2017, which claims priority to U.S. Provisional Patent Application No. 62/319,553, filed Apr. 7, 2016, all of which are incorporated herein by reference in their entireties.

### II. FIELD OF THE INVENTION

The present invention relates to methods and kits for detecting Myo/Nog cells in human adipose tissue, as well as therapeutic use thereof.

### III. BACKGROUND

Myo/Nog cells are characterized by their expression of MyoD mRNA, noggin (Nog), and the G8 antigen. While Myo/Nog cells express a marker of mesenchymal stem cell lineage (CD 105), they are unique in that they produce noggin (Nog). Such cells were originally discovered in chick embryos. MyoD is a protein that has a major role in regulating muscle differentiation and development and acts principally as a transcription factor. MyoD belongs to a greater family of proteins known as myogenic regulatory factors (MRFs). The structure of MyoD is that of the basic helix-loop-helix family. Noggin protein (Nog) is a protein that, like MyoD, is involved principally in the development of body tissues, including nerve tissue, muscle tissue, and bone structure. Amino acid sequence of Nog has remained highly conserved across multiple species. The G8 antigen is an antigen that is recognized by the G8 monoclonal antibody (mAb).

Myo/Nog cells are theorized to be involved in embryonic development. However, mounting evidence has suggested that Myo/Nog cells are present in some adult tissues. Myo/Nog cells were reported as being found in human skin cells in Gerhart et al., (2012); Exp Dermatol. 2012 21(6): 446-468, hereby incorporated by reference in its entirety. Myo/Nog cells are identified by their expression of mRNA for the skeletal muscle specific transcription factor MyoD, the bone morphogenetic protein (BMP) inhibit Noggin (Nog), and the cell surface molecule (G8 antigen) recognized by the G8 monoclonal antibody. Expression of MyoD is hallmark of Myo/Nog cells commitment to skeletal muscle differentiation, while release of Noggin (Nog) is critical for modulating BMP signaling, morphogenesis and differentiation. The depletion of Myo/Nog cells in the blastocyst results in severe malformations of the body wall, central nervous system, and eyes due to reduced BMP signaling. Accordingly, there is a need to identify sources of Myo/Nog cells in adult humans, and there is a further need to develop therapeutic methods of use for such Myo/Nog cells.

### IV. SUMMARY OF THE INVENTION

The present invention relates to detection of Myo/Nog cells in human adipose tissue and methods of use of such Myo/Nog cells derived from human adipose tissue. The present invention has surprisingly discovered that Myo/Nog cells are present in human adipose tissue, and further that such adipose-tissue derived Myo/Nog cells may possess utility in facilitating wound healing.

Accordingly, in some embodiments, the invention is directed to a method for detecting Myo/Nog cells in human adipose tissue. In some embodiments, the method is directed to the steps of providing a sample containing human adipose tissue, processing said sample to provide a stromal-vascular fraction (SVF), contacting the SVF with a first antibody that specifically binds to an antigen present in Myo/Nog cells, and detecting the bound first antibody. In some embodiments, the method contains the further step quantifying the number of Myo/Nog cells present in a sample.

In other embodiments, the invention is directed to a method for isolating Myo/Nog cells in human adipose tissue. In some embodiments, the method is directed to the steps of providing a sample containing human adipose tissue, processing said sample to provide a stromal-vascular fraction (SVF), contacting the SVF with a first antibody that specifically binds to an antigen present in Myo/Nog cells, and isolating the Myo/Nog cells bound to the first antibody.

In other embodiments, the present invention is directed to a method of promoting tissue regeneration in a subject in need thereof. In some embodiments, the method is directed to the steps of isolating Myo/Nog cells from human adipose tissue and administering said isolated Myo/Nog cells to a subject in need thereof. In some embodiments, the Myo/Nog cells are autologous. In some embodiments, the isolated Myo/Nog cells are administered in a composition. In certain embodiments, the composition may further include a therapeutically effective amount of at least one pharmaceutically active ingredient. In one embodiment, the pharmaceutically active ingredient may be a cytotoxic agent, a corticosteroid, an antibiotic, an immunosuppressant or any tissue regeneration enhancer.

In some embodiments, the Myo/Nog cells derived from adipose tissue are administered topically. In some embodiments, the Myo/Nog cells are administered subcutaneously. In some embodiments, the Myo/Nog cells are administered intravenously or by direct injection into a wound.

In some embodiments, the Myo/Nog cells derived from adipose tissue are administered to a subject having a chronic non-healing wound.

In some embodiments, the Myo/Nog cells derived from adipose tissue are co-administered with stromal vascular fraction (“SVF”) to a subject in need thereof.

In some embodiments, the Myo/Nog cells derived from adipose tissue are co-administered with stem cells to a subject in need thereof. In some embodiments, the stem cells comprise adipose derived stem cells (ADSCs).

In other embodiments, the present invention is directed to medical devices or implants containing Myo/Nog cells derived from human adipose tissue.

In other embodiments, the present invention is directed to kits for detecting Myo/Nog cells in human adipose tissue.

In some embodiments, the present invention is directed to a composition comprising enhanced stromal vascular fraction (“SVF”) having a concentration of Myo/Nog cells that is greater than about 5%.

In some embodiments, the present invention is directed to a composition comprising cryopreserved Myo/Nog cells derived from human adipose tissue.

### V. BRIEF DESCRIPTION OF THE DRAWINGS

FIG. 1A represents background fluorescence in testing set 1. FIG. 1B represents fluorescence of secondary antibodies alone in testing set 1. FIG. 1C represents fluorescence of secondary antibodies coupled with an G8 mAb in testing set 1. FIG. 1D represents background fluorescence in testing set 2. FIG. 1E represents fluorescence of secondary antibodies alone in testing set 2. FIG. 1F represents fluorescence of secondary antibodies coupled with anti-Nog antibody in testing set 2.

FIG. 2 represents a diagram of the isolation procedure for the stromal vascular fraction (SVF) from human adipose tissue. Adipose specimens obtained from a human are washed, filtered, and incubated with collagenase for one hour. After centrifugation, the fat and aqueous layers are removed and the resultant cell pellet is known as the stromal-vascular fraction (SVF).

FIG. 3 represents a diagram of magnetic antibody cell sorting process (MACS). A stromal-vascular fraction (SVF) cell suspension was labeled with G8 monoclonal antibody (an IgM) and an anti-IgM secondary antibody conjugated with magnetic beads. The cell suspension was added to a separation column in a magnetic field. G8 antigen-positive cells were eluted by removing the column from the magnet. Cells were subsequently cultured on gelatin-fibronectin coated surfaces or sampled for flow cytometry.

FIG. 4 represents that Myo/Nog cells are present in the stromal-vascular fraction (SVF) of human adipose tissue. SVF cells cultured on tissue culture dishes for five days were incubated with G8 monoclonal antibody (IgM isotype) and a rhodamine conjugated secondary antibody. Nuclei were stained with dapi. G8-antigen positive Myo/Nog cells were a subpopulation within the SVF cell culture, and are indicated by arrows in the figure.

FIGS. 5A-5D represent the detection of Myo/Nog cells in three successive generations of SVF cells. FIG. 5A represents the first generation, FIG. 5B represents the first successive generation, FIG. 5C represents the second successive generation, and FIG. 5D represents the third successive generation. The SVF cells were fluorescently labeled with antibodies to G8 and noggin. Nuclei were stained with dapi. G8 and noggin co-localized to a subpopulation of SVF in all generations, which are indicated by arrows in the figures.

FIGS. 6A-6B represent an analysis of noggin (Nog) and CD 105 expression in Myo/Nog cells by flow cytometry analysis. G8-antigen positive cells were present as 3.5±0.9% of the stromal-vascular fraction (SVF) population that underwent MACS. FIG. 6A represents anti-noggin antibody bound only the G8-positive population. The G8-negative population followed the same counter as the control group of adipose-derived stem cells (ADSCs). FIG. 6B represents anti-CD 105 antibody bound cells in both populations. The G8-negative population was more intensely labeled for CD 105 than the G8-positive population.

### VI. DETAILED DESCRIPTION

Myo/Nog cells exist at low frequency in several different tissues, but have not been previously reported as being present in human adipose tissue. Adipose tissue represents a new and preferable source for Myo/Nog cells because it is already a source of stem cells used for therapeutic applications approved by the U.S. Food and Drug Administration (“FDA”). Furthermore, the processing of the stromal vascular fraction (“SVF”) containing Myo/Nog cells can be isolated in a minimally manipulated method. For therapeutic applications of Myo/Nog cells derived from adipose tissue, a subject may be able to self-donate adipose tissue, e.g. from a simple liposuction procedure. Thus, in some embodiments, the Myo/Nog cells which are administered to an individual are derived from that same individual, i.e. they are autologous. Alternatively, they may be heterologous in circumstances where the Myo/Nog cells are derived from one patient and administered to another patient. As such at least one broad aspect of the present invention is directed to methods of isolating adipose tissue derived Myo/Nog cells from its original environment and administering them to a human subject in need for therapeutic purposes such as improving wound healing.

Myo/Nog cells may react to perturbation in homeostasis in multiple tissues other than human adipose tissue. Without wishing to be bound by theory, it is believed that the propensity of My/Nog cells to respond to wounding reflects, in part, their innate capacity for migration and expression of muscle proteins, particularly skeletal muscle. When removed from embryonic and fetal tissues and cultured in serum-free medium, they translate MyoD mRNA and undergo terminal skeletal muscle differentiation. Myo/Nog cells do not appear to translate MyoD mRNA or synthesize sarcomeric proteins under homeostatic conditions in vivo, however, when activated by apoptotic cells, epidermal abrasion, tumorigenesis, or incisions made in embryonic lens tissue, Myo/Nog cells rapidly increase in number and migrate to the wounded area. The effect of Myo/Nog depletion on the accumulation of myofibroblasts in anterior lens tissue was tested in vitro in Gerhart et al., (2014); PLoS ONE 9(4): e95262, hereby incorporated by reference in its entirety.

In some embodiments, the invention is directed to a method for detecting Myo/Nog cells in human adipose tissue. In some embodiments, the method is directed to the steps of providing a sample containing human adipose tissue, processing said sample to provide a stromal-vascular fraction (SVF), contacting the SVF with a first antibody that specifically binds to an antigen present in Myo/Nog cells, and detecting the bound first antibody. In some embodiments, the first antibody is the G8 monoclonal antibody. Characterization and synthesis of the G8 monoclonal antibody is described in Gerhart et al., (2001); J. Cell. Biol. 155, 381-391, hereby incorporated by reference in its entirety. The G8 monoclonal antibody recognizes a surface antigen specifically expressed in cells that express MyoD mRNA in the epiblast and fetal organs. The G8 monoclonal antibody is a IgM kappa antibody. Use of the G8 monoclonal antibody to detect surface antigens in MyoD-positive cells can be found at, for example, Gerhart et al., (2006), 175(2): 283-292, hereby incorporated by reference in its entirety, and Gerhart et al., (2004), J. Cell. Biol. 164(5): 739-746, hereby incorporated by reference in its entirety. In some embodiments, the first antibody is an anti-Nog antibody. In further embodiments, the detecting step further involves contacting the first antibody with a detectably-labeled secondary antibody. In some embodiments, the detecting step involves contacting the first antibody with a labeled substrate, such as in a competitive immunoassay. In further embodiments, the method contains the further step quantifying the number of Myo/Nog cells present in a sample. In some embodiments, the quantifying step includes a flow cytometry analysis.

In some embodiments, the invention is directed to a method for isolating Myo/Nog cells in human adipose tissue. In some embodiments, the method contains the steps of providing a sample containing human adipose tissue, processing said sample to provide a stromal-vascular fraction (SVF), contacting the SVF with a first antibody that specifically binds to an antigen present in Myo/Nog cells, and isolating the Myo/Nog cells bound to the first antibody. In some embodiments, the first antibody is the G8 monoclonal antibody. In some embodiments, the first antibody is an anti-Nog antibody. In some embodiments, the isolating step includes magnetic cell sorting techniques. In some embodiments, the Myo/Nog cells are isolated from the SVF based on lower level of CD 105 expression than other cells located in the SVF.

In some embodiments, the yield of Myo/Nog cells isolated from stromal-vascular fraction (SVF) is about 1%, about 2%, about 3%, about 4%, about 5%, or greater than about 5%. In some embodiments, the yield of Myo/Nog cells isolated from stromal-vascular fraction (SVF) is between 1% and 2%, between 1% and 3%, between 1% and 4%, between 1% and 5%, between 2% and 3%, between 2% and 4%, between 2% and 5%, between 3% and 4%, between 3% and 5%, between 4% and 5%, less than 1%, or greater than 5%, and any intervening range therein.

In some embodiments, the present invention is directed to a method of promoting tissue regeneration in a subject in need thereof. Myo/Nog cells derived from human adipose tissue may respond to cells undergoing apoptosis and/or tissue wounding by rapidly expanding and/or proliferating and migrating to the site of injury. Myo/Nog cells regulate the activity of bone morphogenetic proteins (BMPs) that play a key role in controlling epidermal homeostasis, hair follicle growth, melanogenesis, wound healing and tumorigenesis. Tissue regeneration may include the renewal, re-growth, or restoration of a body or a bodily part, tissue, or substance after injury or as a normal bodily process. Tissue regeneration is a subtype of wound healing wherein there is restoration of the damaged tissue to its original state, as opposed to wound healing characterized by fibrotic tissue buildup, e.g. by collagen deposition. In some embodiments, the subject is mammalian. In some embodiments, the subject is human.

In some embodiments, the method is directed to a method of administering Myo/Nog cells derived from adipose tissue to a subject having a wound. In some embodiments, the wound is a chronic wound. The wound may be a cutaneous tissue wound. Chronic wounds such as pressure ulcers, diabetic wounds, and venous stasis ulcers share common traits that Myo/Nog cells derived from adipose tissue may prove particularly useful to treat, chiefly that they are characterized by not being able to contract and become smaller. Myo/Nog cells derived from adipose tissue may be able to enhance the wound contracture process when administered to a patient having a chronic wound. Additional types of wounds that Myo/Nog cells may be used to treat include non-healing wounds that arise from surgical intervention, e.g. surgical resection of tumor tissue.

In some embodiments, the method is directed to the steps of isolating Myo/Nog cells from human adipose tissue and administering said isolated Myo/Nog cells to a subject in need thereof, e.g. in a composition prepared from said isolated Myo/Nog cells. In some embodiments, the isolating step includes the steps of providing a sample containing human adipose tissue, processing said sample to provide a stromal-vascular fraction (SVF), contacting the SVF with a first antibody that specifically binds to an antigen present in Myo/Nog cells, and isolating the Myo/Nog cells bound to the first antibody. In some embodiments, the first antibody is an G8 monoclonal antibody. In some embodiments, the first antibody is an anti-Nog antibody. In some embodiments, the isolating step includes magnetic cell sorting techniques. In some embodiments, the administering step further includes co-administration with a stem cell or growth factor, e.g. adipose derived stem cells (ADSCs).

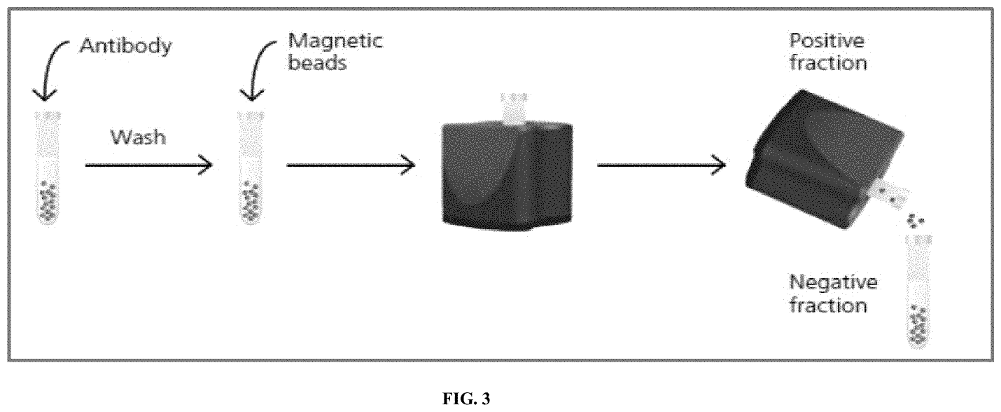

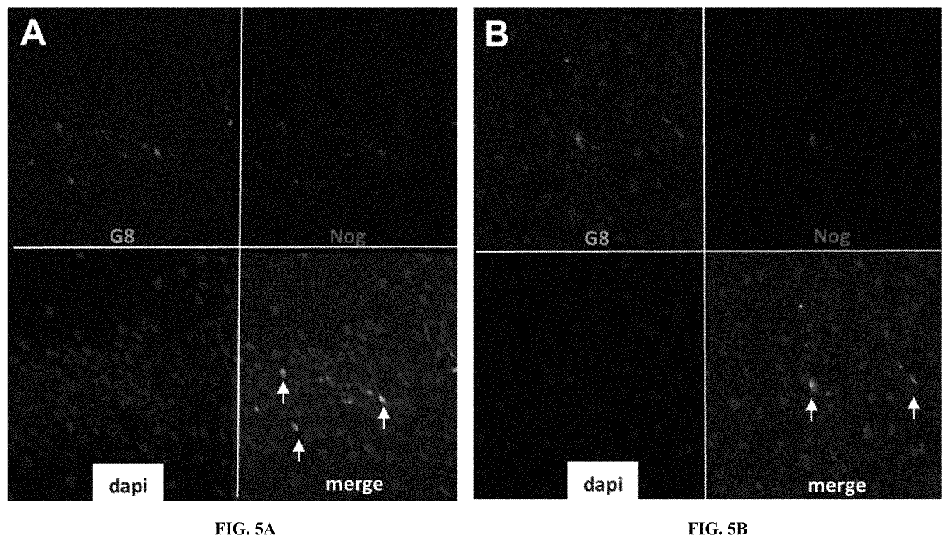

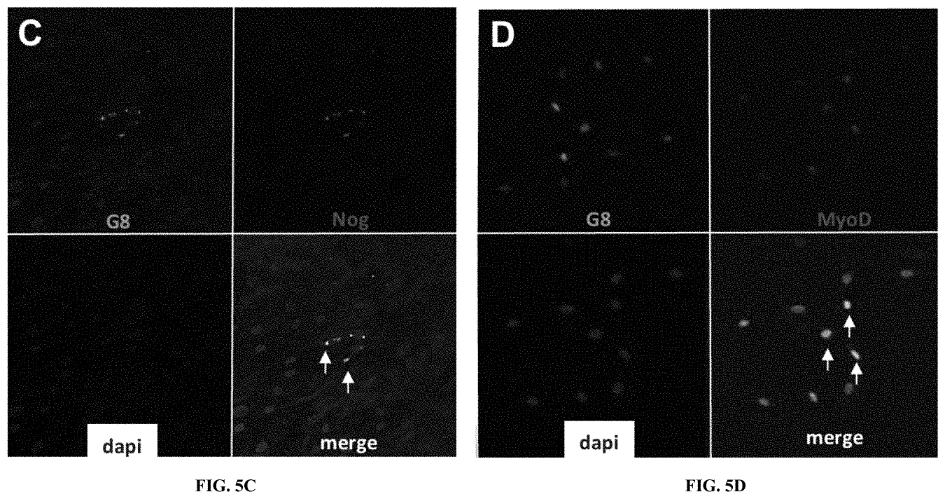

Myo/Nog cells are essential for skeletal muscle formation in the embryo. They are predicted to facilitate muscle regeneration due to their demonstrated ability to promote myoblast differentiation. Accordingly, in some embodiments, administration of Myo/Nog cells derived from human adipose tissue to a subject in need thereof may result in growth or regeneration of skeletal muscle tissue. Without wishing to be bound by theory, this is believed to result from MyoD activity, as illustrated in FIGS. 5A-5D, G8-antigen positive Myo/Nog cells in SFV cultures derived from human adipose tissue continued to be committed to skeletal muscle lineage in mixed cultures of SFV cells, as evidenced through labeling with anti-MyoD antibody. Thus, Myo/Nog cells which are present in human adipose tissue, including adult human adipose tissue, remain viable and retain their identity of skeletal muscle progenitor cells even after isolation by magnetic antibody cell sorting (MACS, shown in FIG. 3), and rearing in culture through several generations in the mixed cellular environment of stromal-vascular fraction (SVF).

The administering step may include methods known in the art. Myo/Nog cells derived from human adipose tissue may be administered in a number of ways depending on whether local or systemic treatment is desired, and on the area to be treated. In a preferred embodiment, the Myo/Nog cells are administered directly to a wounded area or an area surrounding a wound. The administration would preferably be either by direct injection or by topical administration. Administration may be topically, including ophthalmically, vaginally, rectally, intranasally, by inhalation, or parenterally, for example by intravenous drip, subcutaneous, intraperitoneal or intramuscular injection. Injectables can be prepared in conventional forms, either as liquid solutions or suspensions, solid forms suitable for solution of suspension in liquid prior to injection, or as emulsions. Suitable topical forms may include, e.g. ointments; lotions; creams; gels, drops; suppositories; sprays; liquids; powders; sprayable liquids; liquids that may be applied using a roll-on device; lacquers; and sustained release matrices of transdermal delivery devices such as patches. Conventional pharmaceutical carriers, aqueous, powder or oily bases, thickeners and the like may be necessary or desirable. The disclosed compositions can be administered, for example, in a microfiber, polymer, nanosphere, aerosol, lotion, cream, fabric, plastic, tissue engineered scaffold, matrix material, tablet, implanted container, powder, oil, resin, wound dressing, bead, microbead, slow release bead, capsule, injectables, intravenous drips, pump device, silicone implants, or any bio-engineered materials.

In some embodiments, the Myo/Nog cells derived from human adipose tissue are co-administered with another composition. In some embodiments, the Myo/Nog cells are co-administered with stromal vascular fraction (“SVF”) that is derived from human adipose tissue. In some embodiments, the Myo/Nog cells are combined with SVF into a single composition and then administered to a patient. Ideally, such a composition would amount to enriched SVF, with a greater than natural percentage of Myo/Nog cells found in SVF. For example, while Myo/Nog cells may be found in SVF at a rate of about 2% to about 5% as disclosed herein, Myo/Nog cells can be combined with SVF to create a composition that is greater than 5% Myo/Nog cells, for example, between about 5% and about 10%, between about 5% and about 15%, between about 10% and about 15%, between about 10% and about 20%, between about 15% and about 20%, between about 10% and about 25%, between about 15% and about 25%, between 20% and about 25%, and greater than about 25% Myo/Nog cells, e.g. greater than 30%, greater than 35%, greater than 40%, greater than 45%, greater than 50%. These compositions of “enriched” SVF, containing a percentage of Myo/Nog cells higher than what is naturally found, may have enhanced healing or restorative properties than either isolated Myo/Nog cells individually or natural SVF. In other embodiments, the composition may include a therapeutically effective amount of at least one pharmaceutically active agent. In one embodiment, the pharmaceutically active agent may be a cytotoxic agent, a corticosteroid, an antibiotic, an immunosuppressant, cytokine secreting cell or any tissue regeneration enhancer or any combinations thereof.

In one embodiment, the composition may further include conditioned media derived from enriching SVF with or without Myo/Nog cells. In one embodiment, the conditioned media further contains cell lysates or cytokines derived therefrom each alone and/or in combination with each other and/or with other agents including active and/or inactive pharmaceutical agents.

Accordingly, in some embodiments, the present invention is directed to compositions containing Myo/Nog cells derived from human adipose tissue and methods of use administering said compositions. The compositions may include isolated Myo/Nog cells derived from human adipose tissue, or purified Myo/Nog cells derived from human adipose tissue. The compositions may include Myo/Nog cells derived from stromal vascular fraction (“SVF”). The Myo/Nog cells derived from human adipose tissue may be autologous, i.e. derived from the same individual from which the Myo/Nog cells are to be administered. The compositions may further include pharmaceutically acceptable carriers or excipients. In some embodiments, the composition may further contain thickeners, diluents, buffers, preservatives, surface active agents and the like in addition to the pharmaceutically acceptable carriers and the Myo/Nog cells derived from human adipose tissue.

In some embodiments, the present invention is directed to medical devices or implants containing (e.g. coated with) with Myo/Nog cells derived from human adipose tissue or compositions thereof. Non-limiting examples of medical implants include: limb prostheses, breast implants, penile implants, testicular implants, artificial eyes, facial implants, artificial joints, heart valve prostheses, vascular prostheses, dental prostheses, facial prosthesis, tilted disc valve, caged ball valve, ear prosthesis, nose prosthesis, pacemakers, cochlear implants, and skin substitutes. These medical devices may provide particular utility for sustained release of Myo/Nog cells derived from human adipose tissue and directed application to areas where tissue regeneration is needed.

In at least one embodiment, a method for improving wound healing or decreasing wound failure in a surgical patient in need thereof comprising administering to the patient a therapeutically effective amount of one or more compositions comprising Myo/Nog cells, SVF and/or conditioned media derived therefrom, with or without a pharmaceutically active agent to improve wound healing.

In some embodiments, the present invention is directed to kits for detecting Myo/Nog cells in human adipose tissue. The kits may contain reagents for detecting Myo/Nog cells in human adipose tissue. The kits may contain, for example, first antibodies that specifically bind to one or more Myo/Nog antigens, e.g. G8 monoclonal antibodies (mAbs) or anti-Nog antibodies. The antibodies may be monoclonal, polyclonal, chimeric, recombinant, or antibody fragments. The kit may include reagents that bind directly or indirectly to the antibodies that specifically bind to one or more Myo/Nog antigens. The reagents may be e.g. bound tracer, including detectably labeled standards/tracer, or may be secondary antibodies, including detectably labeled secondary antibodies. The first antibodies may be immobilized on a substrate, e.g. in a sandwich assay. In such embodiments, there may be a second antibody that binds to a different epitope on the Myo/Nog antigen(s), and that antibody may be detectably labeled. In other embodiments, the first antibodies may be free, and/or may bind to secondary antibodies that are coated on a substrate, such as in a competitive enzyme immunosorbent assay (EIA). These captured antibodies may bind competitively to labeled standards/tracer. In some embodiments, the labeled standards may be labeled with, e.g. biotin, and the kits may further include streptavidin-linked enzymes, e.g. horseradish peroxidase (HRP). Such embodiments may produce a soluble colored product that is detectable, e.g. a colorimetric assay. In other embodiments, the antibodies may be free, and competitively bind to radiolabeled standard peptides such as in a radioimmunoassay (RIA). The kits may further include diluents or and/or preservatives. The present invention also covers methods of use of these kits.

Myo/Nog cells derived from human adipose tissue, as shown in Example 1 infra, are capable of surviving freezing and thawing after purification. Thus, Myo/Nog cells are expected to survive cryopreservation. Accordingly, in some embodiments, the present invention is directed to a composition comprising cryopreserved Myo/Nog cells derived from human adipose tissue.

The following non-limiting examples further illustrate certain aspects of the invention.

### VII. EXAMPLES

### 1. Identification of Myo/Nog Cells in Human Adipose Tissue

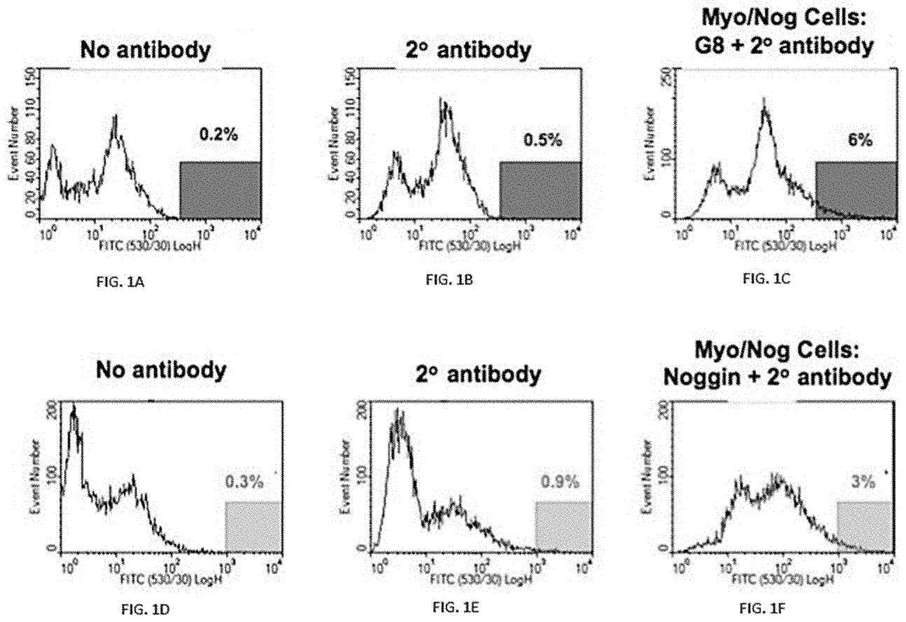

Adipose tissue was donated by individuals undergoing liposuction. Fresh tissue was processed from the donated adipose tissue to remove red blood cells according to a standard protocol for isolating adipose-derived stem cells (ADSCs). The resultant stromal-vascular fraction (SVF) was utilized to determine whether or not Myo/Nog cells were present in SVF, and if so, the size of the population. Further, it was determined whether or not magnetic sorting could be utilized to isolate Myo/Nog cells if present, and finally whether or not isolated Myo/Nog cells could survive in culture. SVF cells split into two testing populations. The first testing population (testing set 1) was tested with the G8 monoclonal antibody (mAb), which is known to be a specific marker for Myo/Nog cells and a fluorescent secondary antibody or no antibodies to determine the level of background fluorescence. The second testing population was tested with an anti-Nog antibody (specific for Noggin), and a fluorescent secondary antibody or no antibodies to determine the level of background fluorescence. Results are indicated in FIGS. 1A-1F.

Briefly, in testing set 1, no antibodies (control) exhibited a background fluorescence of 0.2%, secondary antibodies alone exhibited a fluorescence of 0.5% (non-specific binding), and secondary antibodies coupled with the G8 mAb exhibited fluorescence of 6%, up from the background of 0.2%. In testing step 2, no antibodies (control) exhibited a background fluorescence of 0.3%, secondary antibodies alone exhibited a fluorescence of 0.9% (non-specific binding), and secondary antibodies coupled with the anti-Nog antibody exhibited a fluorescence of 3.0%, up from 0.3%. Such results indicate two measures; the first is a positive identification of Myo/Nog cells in human adipose tissue, and the second is that the G8 mAb is likely superior to the anti-Nog antibody (for detection purposes), although the anti-Nog antibody still showed measurable detection and is considered embodied by the present invention. Utilization of both antibodies may provide superior results.

Flow cytometry analyses indicated that the cells labeled with the G8 mAb and anti-Nog antibody constituted a minor but significant population within the SVF isolate. Myo/Nog cells labeled with the G8 mAb were successfully isolated by magnetic cell sorting. Briefly, of 2,387,500 total cells, Myo/Nog cells represented 87,383 or 3.7% that were successfully isolated by magnetic cell sorting. Other cells represented 2,300,117 or 96.3%. In a first experiment, unsorted SVF cells that had been stored frozen were thawed and placed in culture in a petri dish. Labeling with the G8 mAb indicated that Myo/Nog cells had survived freezing and subsequent thawing. The Myo/Nog cells attached to the petri dish they were cultured in. These Myo/Nog cells grew under conditions that were developed for adipose-derived stem cells. In a second experiment, unsorted SVF cells were grown and replated twice in different culture dishes. Myo/Nog cells were still present in these “passaged” cultures. These findings indicate that Myo/Nog cells are present in human adipose tissue and remain viable despite storage in a freezer, passaging of SVF cells in culture, and isolation by magnetic cell sorting.

2. Further Identification of the unique MyoD and Noggin-expressing lineage (Myo/Nog) in Human Adipose Tissue

Eight adipose specimens were obtained from healthy adult volunteers following an IRB-approved protocol and treated to isolate the stromal-vascular fraction (SVF). Isolates were cultured through several generations, imaged using fluorescence microscopy for G8 and noggin, separated by magnetic-activated cell sorting (MACS) for G8, and tested for noggin and CD 105, a mesenchymal stem cell marker, utilizing flow cytometry. The following methodologies were utilized for this Example.

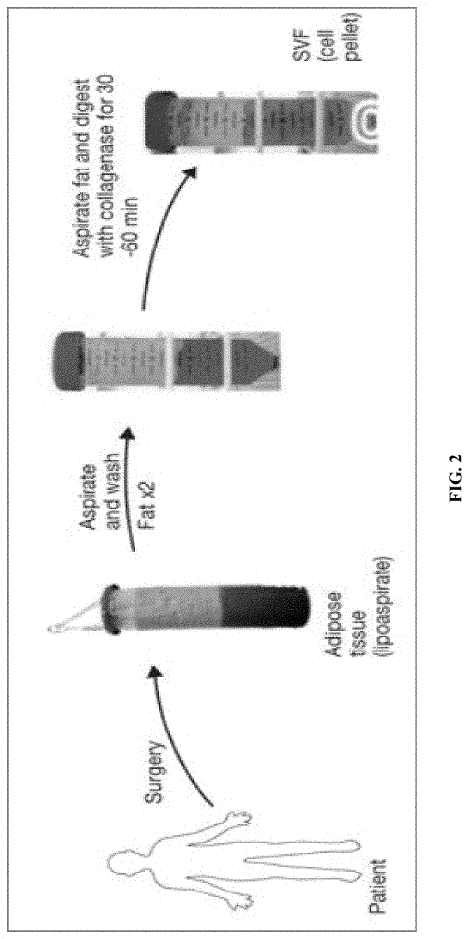

Adipose specimens were obtained from adult volunteers following an IRB-approved protocol. Solid samples were minced with scissors to 3 mm size. Both liposuction aspirate and minced fat were rinsed with phosphate-buffered saline and incubated with 0.1% collagenase for one hour at 37° C. with vigorous shaking. This is illustrated in FIG. 2. After centrifugation for 10 minutes at 1000 g, the lipid and aqueous layers were discarded and the residual cell pellet was treated with water to lyse erythrocytes, washed with M-199 medium, and re-centrifuged. The final cell pellet—the stromal vascular fraction (SVF)—was re-suspended with M-199 containing 10% fetal bovine serum (FBS) and antibiotics/antimycotics and incubated at 37° C. in 5% CO₂ in culture plates or flasks coated with 0.1% gelatin and 5% human serum fibronectin. Prior to plating, a small aliquot of the cell solution was counted and assessed for viability utilizing the Nucleocounter-200. After 24 hours, the medium was changed to remove non-adherent cells.

Three SVF specimens were plated in gelatin-fibronectin coated 6-well plates with removable glass coverslips through three passage generations (P0, P1, P2). A fourth SVF fraction was plated on 35 mm uncoated tissue dishes and passaged once before fixation. Cells were grown in M-199 containing 10% fetal bovine serum (FBS) and antibiotics/antimycotics.

With the exception of one specimen, SVF was grown on coated T75 flasks for 6-8 days until confluent. Cells were treated with trypsin, centrifuged for five minutes at 1000 g, and re-suspended in 0.5% bovine serum albumin (BSA) solution. A small aliquot was taken for counting and viability assay utilizing the Nucleocounter-200. The cell suspension and one fresh SVF isolate were then incubated with G8 monoclonal antibody (mAb) at 1:10 dilution for 45 minutes at room temperature. This was followed by several washings with BSA. The cell pellet was incubated with 200 μL MACS beads according to the manufacturer recommendation for 15 minutes at 4° C. The suspension was then applied to a mini-MACS column and three washes with 500 μL BSA were performed with the column in the magnetic field to remove G8-negative cells. This process is indicated in FIG. 3. The resultant G8-positive and G8-negative fractions were assessed again with the Nucleocounter-2000 to determine yield, Myo/Nog percentage in SVF, and viability.

G8-positive and G8-negative cells were cultured on gelatin-fibronectin coated dishes in M199 containing 10% fetal bovine serum (FBS). After reaching 80% confluence, culture medium was changed. Muscle differentiation was encouraged by growth in serum-free M199 medium for 5-7 days followed by immediate fixation. Sorted aliquots were also utilized for flow cytometry characterization of expression of noggin (Nog) and CD 105.

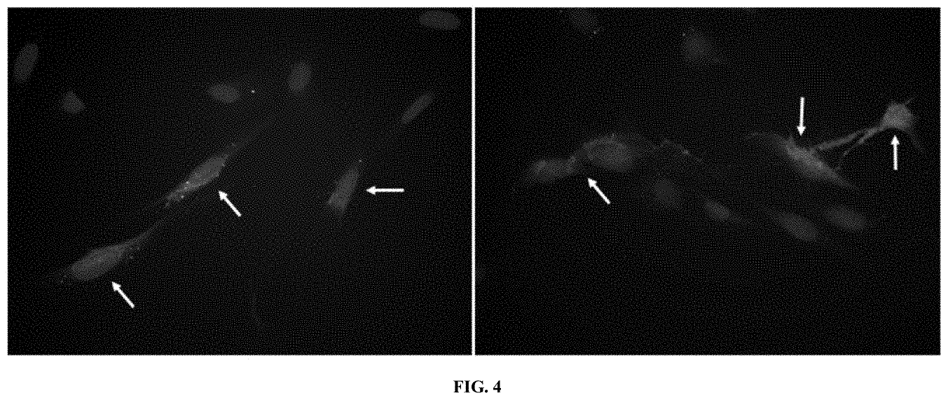

The SVF population was screened for Myo/Nog cells by immunofluorescence localization of the G8 protein and MACS. Freshly isolated SVF cells passaged once and then grown for five days contained a minor subpopulation of cells that bound to the G8 monoclonal antibody (FIG. 4). Myo/Nog cells, which are characterized by co-localization of G8 antigen and noggin (Nog), were also present in SVF cultures following three passages of cell expansion (FIGS. 5A-5D). Labeling with an anti-MyoD antibody revealed that G8-antigen positive Myo/Nog cells continued to be committed to skeletal muscle lineage in mixed cultures of SVF cells.

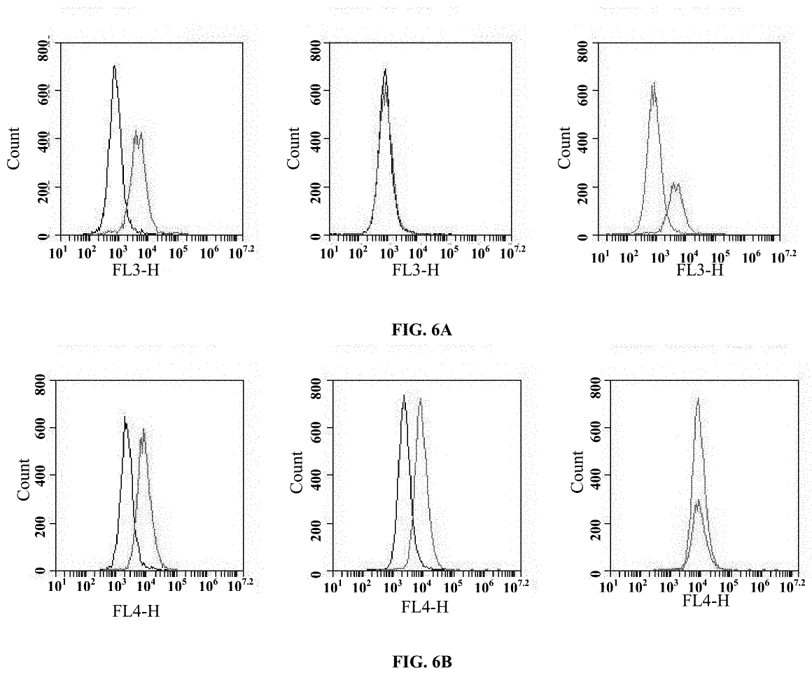

Isolation of G8-antigen positive cells by MACS revealed that Myo/Nog cells constituted 3.7% (S.D. 1.0%) of the SVF population in four individual specimens, Table 1 below. Flow cytometry analysis confirmed that noggin (Nog) was present specifically in the G8-antigen positive population compared to concurrently isolated adipose-derived stem cells (ADSCs), whereas CD 105, a marker of mesenchymal stem cell lineage, was present in all cells (FIGS. 6A-6B).

TABLE 1
Percent of Myo/Nog cells in SVF. SVF cells were incubated with
G8 monoclonal antibody and isolated by MACS. The percent of
G8-positive cells was determined by automatic cell counting.

| Sample No. (ID) | Yield (%) |
|---|---|
| 1 (id 120415p) | 2.4% |
| 2 (id 021816p) | 4.9% |
| 3 (id 022516f) | 3.6% |
| 4 (id 032216p) | 3.8% |
| Mean Yield | 3.7% |
| Standard Deviation | 1.0% |

It will be understood by those of skill in the art that numerous and various modifications can be made without departing from the scope and spirit of the present invention. Therefore, it should be understood that various embodiments of the invention described herein are illustrative only and not intended to limit the scope of the invention. All references cited herein are incorporated by reference in their entirety.

1. A method of detecting Myo/Nog cells in a sample, the method comprising:
(a) providing a sample comprising human adipose tissue,
(b) processing said sample to provide a stromal-vascular fraction (SVF),
(c) contacting said SVF with a first antibody that specifically binds to an antigen present in Myo/Nog cells, and
(d) detecting the bound first antibody.

2. The method of claim 1, further comprising:
(e) quantifying the number of Myo/Nog cells present in a sample.

3. The method of claim 1, wherein the first antibody is the G8 monoclonal antibody.

4. The method of claim 1, wherein the first antibody is an anti-Nog antibody.

5. The method of claim 1, wherein the detecting step comprises contacting the bound first antibody with a secondary reporter antibody.

6. The method of claim 1, wherein the detecting step comprises contacting the first antibody with a labeled substrate.

7. A method of regenerating tissue in a subject in need thereof, the method comprising:
(a) isolating Myo/Nog cells from human adipose tissue,
(b) administering to said subject in need thereof said a composition comprising said isolated Myo/Nog cells.

8. The method of claim 7 wherein the composition further comprises pharmaceutically acceptable carriers or diluents.

9. The method of claim 7 wherein the administration is topical.

10. The method of claim 7 wherein the administration is via one of a scaffold or implant.

11. A method of isolating Myo/Nog cells in sample, the method comprising:
(a) providing a sample comprising human adipose tissue,
(b) processing said sample to provide a stromal-vascular fraction (SVF),
(c) contacting said SVF with a first antibody that specifically binds to an antigen present in Myo/Nog cells, and
(d) isolating cells from said SVF that are bound to said first antibody.

12. The method of claim 11 wherein the isolating step comprises magnetic cell sorting.

13. A kit for detecting Myo/Nog cells in a sample, the kit comprising:
(a) a first antibody that specifically binds to one or more Myo/Nog antigens; and
(b) a reagent that binds directly or indirectly to the first antibody.

14. The kit of claim 13 wherein the first antibody is immobilized on a substrate.

15. The kit of claim 13 further comprising a secondary antibody.

16. The kit of claim 15 wherein the secondary antibody is detectably labeled.

17. The kit of claim 15 wherein the secondary antibody is immobilized on a substrate.

18. A composition comprising enriched SVF having a concentration of Myo/Nog cells that is greater than about 5%.

19. The composition of claim 18, further comprising a therapeutically effective amount of a pharmaceutically active ingredient selected from the group consisting of a cytotoxic agent, a corticosteroid, an antibiotic, an immunosuppressant, a cytokine secreting cell, a tissue regeneration enhancer and combinations thereof.
